(19) United States
(12) Patent Application Publication
(10) Pub. No.: US 2013/0129709 A1
(43) Pub. Date: May 23, 2013

(54) Fraction of Proteins and Peptides Derived from Egg White and Protein Derived From Egg White and Use Thereof as Anti Listeria Agent

(71) Applicants: Sophie Rehault-Godbert, Tours (FR); Yves Nys, Saint Laurent en Gatines (FR); Joël Gautron, Saint Cyr sur Loire (FR); Valérie Labas, Tours (FR); Emmanuelle Helloin, Nouzilly (FR); Cindy Slugocki, Semblancay (FR)

(72) Inventors: Sophie Rehault-Godbert, Tours (FR); Yves Nys, Saint Laurent en Gatines (FR); Joël Gautron, Saint Cyr sur Loire (FR); Valérie Labas, Tours (FR); Emmanuelle Helloin, Nouzilly (FR); Cindy Slugocki, Semblancay (FR)

(73) Assignee: INSTITUT NATIONAL DE LA RECHERCHE AGRONOMIQUE

(21) Appl. No.: 13/701,473

(22) PCT Filed: Jun. 1, 2011
(86) PCT No.: PCT/EP11/59126

§ 371 Date: Feb. 5, 2013

(30) Foreign Application Priority Data
Jun. 4, 2010 (FR) 1054388

(51) Int. Cl. C07K 14/77; A01N 37/18

(52) U.S. Cl. CPC C07K 14/77; A01N 37/18
USPC 424/94.61; 530/367; 514/2.4

(57) ABSTRACT

The invention relates to a fraction of peptides and/or proteins that are derived from egg white and that are capable of binding heparin, for its anti-Listeria monocytogenes action. The invention also relates to a molecule of sequence SEQ ID no. 1 for its antimicrobial action, and in particular anti-Listeria monocytogenes action.

### FIELD OF THE INVENTION

The invention relates to a fraction of proteins and/or peptides that are derived from egg white and present an anti-Listeria monocytogenes activity. The invention also relates to a specific molecule derived from egg white, namely the OVAX protein, for its antimicrobial action and, more specifically, for its targeted action against Listeria monocytogenes.

The invention finds applications notably in the agri-food field, for example during the preparation of food products for current consumption such as deli products and cheeses, in order to eliminate all lanes of Listeria monocytogenes in these products. The invention also finds applications in the pharmaceutical field, for example for the preparation of drugs intended to treat and/or prevent illnesses related to Listeria monocytogenes, such as Listeriosis.

### STATE OF THE ART AND PROBLEMS ENCOUNTERED

Listeria monocytogenes is a bacterium present in soil, vegetation, water, waste waters, silage products and human and animal fecal matter. Some animals may unknowingly be carriers of the bacteria, without being sick. These healthy carriers can be the source of contamination in food, such as milk and meat, and derived products. In particular, this bacterium is regularly found in deli products and raw-milk cheeses. Listeria monocytogenes can thus be the source of an illness called Listeriosis, related to food-poisoning. Listeriosis can have serious consequences, especially for pregnant women, elderly people and people with weakened immune systems.

Up to now it has not been possible to guarantee the absence of Listeria monocytogenes in a food item, except through strict hygiene regulations and thoroughly cooking food and foodstuffs likely to be carriers of the germ.

Listeria monocytogenes is known to be sensitive to certain antibiotics, in particular ampicillin and amoxicillin. Insofar as the people who must be treated for Listeriosis in the event of infection are vulnerable people, it is known to treat them with a combination of antibiotics, in particular penicillin, streptomycin and sulfonamides. This treatment can be extended with lactams.

Despite this aggressive, and often long, course of treatment, results remain random and above all dependent on the condition of the patient's immune system.

In addition, insofar as the bacteria's resistance, even multi-resistance, to antibiotics is more and more frequent, it is important to find an alternative to this treatment in order to overcome such an occurence and/or to prevent it developing for Listeria monocytogenes.

### DESCRIPTION OF THE INVENTION

The invention seeks to overcome at least partially at least one of the problems described above, by providing a new compound likely to act against Listeria monocytogenes.

To achieve this, the invention proposes using a molecule having antimicrobial activity directed against Listeria monocytogenes. The molecule according to the invention is derived from a protein present in eggs, chicken eggs in particular, and more specifically in the egg white, known to comprise many proteins and peptides that have effects against bacteria, and in particular enzymes such as lysozyme. The protein of interest, hereafter called OVAX for “ovalbumin-related protein X”, has also been identified in smaller quantities in the egg yolk, egg shell, vitelline membrane etc.

The amino-acid sequence corresponding to OVAX has been identified, so that it is possible to use the native protein, i.e. isolated from the chicken egg, and also the recombinant protein.

The invention has also made it possible to demonstrate that a fraction of peptides and/or proteins derived from chicken egg white and capable of binding heparin-sepharose (or heparin coupled with any other resin), presents an anti-Listeria monocytogenes activity. The invention has made it possible to identify the peptides and proteins of this fraction, fifteen in number, which comprise in particular OVAX, which advantageously represents a proportion of at least 50%, more precisely at least 56%, of this fraction.

Surprisingly, it was noted that the anti-Listeria monocytogenes activity of the OVAX protein and of the fraction according to the invention was preserved after digestion by certain digestive enzymes and notably by trypsin or chymotrypsin. Trypsin is a digestive enzyme present in the gastric juices of humans and mammals, while chymotrypsin is a digestive enzyme present in the pancreas of humans and mammals. Both play a role in digesting proteins contained in the food ingested by human and non-human animals. Such results therefore make it possible to envisage this OVAX molecule and/or the fraction being used in a therapeutic and/or prophylactic food composition that must be ingested by the human or non-human animal, insofar as the anti-Listeria monocytogenes activity is preserved after the action of the trypsin and/or chymotrypsin.

An object of the invention is therefore a molecule of amino-acid sequence SEQ ID No. 1 for its antimicrobial and, more specifically, anti-Listeria monocytogenes, activity.

Sequence SEQ ID No. 1 corresponds to the amino-acid sequence of the OVAX protein.

In effect, the inventors have discovered that the molecule according to SEQ ID No. 1 possesses a particular affinity for heparin, a negatively charged glycosaminoglycan, and consequently could be bound to negatively charged surfaces, such as the lipopolysaccharide of certain Gram-negative bacteria, and the peptidoglycan of certain Gram-positive bacteria (due to the presence of teichoic acid). The binding between at least one of these positively charged sequences and the negatively charged surface of a microorganism leads to destabilization of the wall of said microorganism, leading to its lysis. The molecule according to the invention is therefore capable of inhibiting the growth of such microorganisms.

Thus the invention concerns more particularly the molecule of sequence SEQ ID No. 1 for its anti-Listeria monocytogenes, action, as this is more specifically described below.

The invention also relates to a composition comprising a molecule of sequence SEQ ID No. 1, as active antimicrobial ingredient and in particular as active anti-Listeria monocytogenes ingredient.

The composition according to the invention, comprising the antimicrobial molecule of amino-acid sequence SEQ ID No. 1, can in particular be intended for the production of an anti-Listeria monocytogenes drug. Such a composition can in particular be a solution intended to be ingested, and comprising the required excipients. Otherwise, the composition can be a powder intended to be put into suspension subsequently, or to be packaged as such in the form of gellules.

The composition according to the invention can also be used for producing foodstuffs intended for consumption by human and non-human animals, as an anti-Listeria monocytogenes food additive.

Advantageously, the composition is then used during the production process for the food product, said composition being added into the preparation intended to form the food product, while it is being prepared.

In particular, the composition can be used as a food additive in the preparation of deli products such as pâtés, rillettes, sausages etc. The composition can also be added to milk intended to be used in cheese-making.

Preferably, the composition is added to the preparation after all cooking steps and/or all steps in which said preparation is subjected to temperatures above 60° C.

According to the invention, the concentration of the molecule of sequence SEQ ID No. 1 in the composition can be between 15 μg/mL and 400 μg/mL, and preferably between 20 μg/mL and 100 μg/mL. Even more preferably, the concentration of the molecule of sequence SEQ ID No. 1 in the composition is between 25 μg/mL and 60 μg/mL.

The invention also relates to a fraction of peptides and/or proteins that are derived from egg white and that are capable of binding heparin, for its anti-Listeria monocytogenes action.

‘Derived from egg white’ means that the peptides and proteins present in the fraction according to the invention are all found in egg whites and can be obtained from egg white. Of course, these peptides and proteins may be obtained in other ways than by purification from egg white, in particular by chemical synthesis or produced in recombinant form.

‘Anti-Listeria monocytogenes action’ and ‘action directed against Listeria monocytogenes’ mean that this fraction is capable of inhibiting the growth of this bacterium and/or destroying it.

All the molecules of said fraction are capable of binding heparin. Thus, the fraction according to the invention is likely to comprise all or part of the peptides and/or proteins derived from egg white capable of binding heparin. This fraction can comprise some contaminating ovalbumin molecules, the main protein in egg white, which represents a proportion of more than 50% of the total proteins in egg white, and which has no affinity for heparin.

These peptide and protein molecules were isolated directly from chicken egg white, by affinity chromatography using heparin-sepharose beads, then identified by mass spectrometry.

The fraction according to the invention thus comprises at least one of the molecules from the following peptides and proteins (the GI references below are extracted from the NCBI database, updated in April 2011):
OVAX, of molecular weight approximately 45 kDa, more specifically approximately 44 kDa, of sequence SEQ ID No. 1, and presenting 1 HBS consensus site binding heparin;
a protein similar to the protein MGC82112 (GI: 118083274), of molecular weight approximately 170 kDa and presenting 1 HBS consensus site binding heparin;
Avidin (GI: 45384354), of molecular weight approximately 17 kDa; this molecule does not seem to present any HBS consensus site binding heparin, which suggests that it presents one or more conformational binding sites for heparin;
Lysozyme C (GI: 229157), of molecular weight approximately 14 kDa; this molecule does not seem to present any HBS consensus site binding heparin, which suggests that it also presents one or more conformational binding sites for heparin;
beta-defensin 11 (GI: 49169808), of molecular weight approximately 12 kDa; this molecule does not seem to present any HBS consensus site binding heparin, which also suggests that it presents one or more conformational binding sites for heparin;
the protein TENP (GI: 46048814), of molecular weight approximately 47 kDa; this molecule does not seem to present any HBS consensus site binding heparin, which suggests that it too presents one or more conformational binding sites for heparin;
cyclophilin B (peptidylprolyl isomerase B) (GI: 45382027), of molecular weight approximately 22 kDa and presenting 1 HBS consensus site binding heparin;
the protein Vmo-I (for “vitelline membrane Outer Layer Protein I”) (GI: 576329), of molecular weight approximately 18 kDa and presenting 1 HBS consensus site binding heparin;
ovotransferrin (Conalbumin) (GI: 71274075), of molecular weight approximately 78 kDa and presenting no HBS consensus site binding heparin;
ovocleidin 17 (GI: 31615312), of molecular weight approximately 15 kDa and presenting no HBS consensus site binding heparin, which suggests that it too presents one or more conformational binding sites for heparin;
ovoglycoprotein (GI: 45383093), of molecular weight approximately 22 kDa and presenting no HBS consensus site binding heparin, which suggests that it too presents one or more conformational binding sites for heparin;
ovalbumin (GI: 28566340), of molecular weight approximately 43 kDa and presenting one HBS consensus site binding heparin;
the predicted “plasma protease Cl inhibitor-like” protein (GI: 326920260), of molecular weight approximately 52 kDa and presenting one HBS consensus site binding heparin;
Clusterin (GI: 45382467), of molecular weight approximately 51 kDa and presenting 2 HBS consensus sites binding heparin; and
a predicted “hypothetical protein” protein (GI: 50728948) similar to pleitrophin (growth factor 8 binding heparin) of molecular weight approximately 18.5 kDa and presenting 5 HBS consensus sites binding heparin.

‘Conformational binding site for heparin’ means a specific exposure of positively charged amino acids when the molecule is conformational, and forming a site capable of binding heparin.

The fraction according to the invention comprises at least the molecule of sequence SEQ ID No. 1, which corresponds to the sequence of OVAX as isolated from egg white. Otherwise, or equally, the fraction can comprise the OVAX molecule of sequence SEQ ID No. 2, which corresponds to the known sequence predicted from the bioinformatic analysis of the chicken genome, and which is identified in the NCBI database by reference XP_{—}418984.2, with the name similar to Ovalbumin-related protein Y (Gene Y protein), called OVAXdb in FIG. 3.

The fraction according to the invention can advantageously comprise all the peptides and proteins derived from egg white and capable of binding heparin, as listed above.

In this case, it is interesting to ensure that the molecule of sequence SEQ ID No. 1 and/or SEQ ID No. 2 represents a proportion of at least 50% of the molecules of the fraction.

Such a fraction can then be obtained directly by passing the egg white through heparin-sepharose (or heparin-agarose) type affinity chromatography, and used as such. Indeed, the fraction obtained by this chromatography de facto comprises more than 50%, and even more than 56%, OVAX (FIG. 1, lane 5—HB-EW, stain around 45-50 kDa). It is the quantification of the pixels of the 50 kDa band of the gel relative to the total number of pixels of the well that allowed the quantity of the OVAX molecule in this fraction to be estimated as at least 50%, and even at least 56%, as a proportion. OVAX is also found in the other bands, but since the OVAX is then with other molecules the portion attributable to OVAX cannot be quantified.

Thus, this fraction can be used directly in the agri-food industry or in the medical field, because eggs are foodstuffs that are commonly used by people and whose safety is recognized, except for people with allergies.

Of course, all or part of the molecules of the fraction according to the invention can also be obtained by chemical synthesis or in the form of recombinant proteins, in particular from the corresponding peptide sequence, since the chicken genome has already been completely sequenced.

An object of the invention is also a composition comprising the fraction of peptides and/or proteins according to the invention as active anti-Listeria monocytogenes ingredient. In other words, the fraction of peptides and/or proteins according to the invention can be used as active anti-Listeria monocytogenes ingredient for producing a medicinal, dietary or other composition.

According to the invention, the total concentration of peptides and/or proteins derived from egg white and capable of binding heparin in the composition can be between 15 μg/mL and 400 μg/mL, and preferably between 20 μg/mL and 100 μg/mL. Even more preferably, the concentration of these molecules in the composition is between 25 μg/mL and 60 μg/mL.

‘Total concentration’ means the cumulative concentration of each of the peptides and/or proteins according to the invention in the fraction used to produce the composition.

According to preferred embodiments of the invention, the fraction present in the composition is digested by at least one digestive enzyme, preferably trypsin or chymotrypsin, prior to its use.

The composition based on the fraction of peptides and/or proteins according to the invention can in particular be intended for the production of an anti-Listeria monocytogenes drug. Such a composition can be a solution intended to be ingested, and comprising the required excipients. Otherwise, the composition can be a powder, and in particular egg white powder possibly enriched in the fraction according to the invention, for putting into suspension subsequently, or for packaging in the form of gellules.

The composition according to the invention can also be used for producing foodstuffs intended for animal consumption, human or non-human, as an anti-Listeria monocytogenes food additive.

Advantageously, the composition is then used during the production process for the food product, said composition being added into the preparation intended to form the food product, while it is being prepared.

In particular, the composition can be used as a food additive in the preparation of deli products such as pâtés, rillettes, sausages etc. The composition can also be added to milk intended to be used in cheese-making.

Preferably, the composition is added to the preparation after all cooking steps and/or all steps in which said preparation is subjected to temperatures above 60° C.

### BRIEF DESCRIPTION OF THE FIGURES

FIG. 1 is an SDS-PAGE gel photograph showing the migration of the chicken egg white proteins; thus, one can see trypsin alone (lane T), chymotrypsin alone (lane CT), the egg white proteins as a whole (lane EW), the proteins of the fraction of egg white not binding heparin (lane HUB-EW), the proteins of the fraction of egg white binding heparin (lane HB-EW), the proteins of the fraction of egg white binding heparin after digestion with trypsin (lane HB-EW-T), and the proteins of the fraction of egg white binding heparin after digestion with chymotrypsin (lane HB-EW-CT).

FIG. 2 is a photograph of an SDS-PAGE gel of the various steps of a first mode of purifying OVAX; the first lane (1) represents the egg white; the second lane (2) the fraction eluted from heparin-sepharose; the third lane (3) the unbound fraction of biotin-sepharose; and the fourth lane (4) the peak corresponding to OVAX after molecular sieving.

FIG. 3 is an alignment of amino acid sequences between the OVAX sequence from the NCBI database (OVAXdb, XP_{—}418984.2 GI: 118086485) and the sequence predicted by the Georgia Institute of Technology's Genemark software system (http://exon.gatech.edu/eukhmm.cgi) (OVAXgs) from the nucleotide sequence corresponding to the OVAX gene (reference NW_{—}001471638.1 in the NCBI database).

FIG. 4 consists of two graphs (FIGS. 4A and 4B) respectively showing the antimicrobial activity in liquid medium of Ovalbumin and OVAX obtained by the first purification mode with respect to Listeria monocytogenes. The growth of Listeria monocytogenes was monitored at 600 nm for 20 hours without Ovalbumin/OVAX or with increasing concentrations of Ovalbumin/OVAX.

FIG. 5 consists of five graphs (FIGS. 5A to 5E) showing the anti-Listeria monocytogenes activity, in liquid medium, of different egg white samples, in different concentrations. Thus, FIG. 5A shows the anti-Listeria monocytogenes activity of pure egg white; FIG. 5B shows the anti-Listeria monocytogenes activity of the fraction of egg white not binding heparin; FIG. 5C shows the anti-Listeria monocytogenes activity of the fraction of egg white binding heparin; FIG. 5D shows the anti-Listeria monocytogenes activity of the fraction of egg white binding heparin, after digestion by trypsin; FIG. 5E shows the anti-Listeria monocytogenes activity of the fraction of egg white binding heparin, after digestion by chymotrypsin.

FIG. 6 is a photograph of an SDS-PAGE gel produced after a second OVAX purification mode, the single lane representing the final fraction obtained.

FIG. 7 is a photograph of an agar medium showing the anti-Listeria monocytogenes activity of OVAX obtained by the second purification mode, at different concentrations, with regard to Beta-defensin 11 (positive control) and Ovalbumin (negative control).

### EXPERIMENT 1

1/1—Material

Collecting and Preparing the Egg White.

The egg whites were collected from several eggs from laying hens (Isa-Hendrix, St Brieuc, France) and diluted by ½ in 50 mM Tris-HCl/150 mM NaCl, pH 7.4, buffer.

The sample was gently homogenized and centrifuged at 14,000 g during 10 minutes at 4° C.

The supernatant obtained was frozen until used later. Of course, it is otherwise possible to proceed directly with the purification steps, without prior freezing.

1/2—Methods

1/2.1—Purifying the OVAX.

A heparin-sepharose type chromatography was performed using the “batch” technique according to the instructions of the supplier (GE-Healthcare brand, distributed by Fischer Scientific, W7346E).

0.6 g of heparin-sepharose beads were equilibrated four times in 50 mM Tris-HCl/50 mM NaCl, pH 7.4, buffer in 50 mL Falcon tubes.

15 mL of egg white prepared as described above and 15 mL of 50 mM Tris-HCl/150 mM NaCl, pH 7.4, were added in each tube.

Incubation was carried out, under gentle agitation, for a whole night at 4° C.

At the end of incubation, the proteins without affinity for heparin-sepharose were eliminated by successive washes with a 50 mM Tris-HCl/150 mM NaCl, pH 7.4, buffer and centrifugation of the beads for 5 minutes at 1,500 g at 4° C.

The effectiveness of the washes was checked by measuring absorbency at 280 nm, an absorbency of 0 attesting to the total elimination of these proteins.

The beads were then charged on two 5 mL polypropylene columns (Qiagen, courtaboeuf, France, 34964).

Elution of affine proteins was carried out with a 50 mM Tris-HCl/1M NaCl, pH 7.4, buffer in 1 mL steps on the two columns in parallel (experience has shown that the yield was better by charging two columns rather than one) and monitored by measuring absorbency at 280 nm.

The most concentrated elution fractions were regrouped and dialyzed for 24 hours against the 50 mM Na phosphate/50 mM NaCl, pH 7.4 buffer (PBS) in dialysis membranes (3500 Da exclusion) (Spectrum brand, distributed by Fischer Scientific, 132720).

After dialysis, the sample was centrifuged at 10,000 g for 10 minutes at 4° C. The supernatant was then charged in a 1 mL polypropylene column, in which 1.5 mL of beads of agarose coupled to biotin (Sigma-Aldrich, Saint Quentin Fallavier, France, B0519) had previously been equilibrated in the PBS buffer.

The column's unbound material was recovered and concentrated on a concentration cell with 3500 Da exclusion (Millipore, UFC900324). This step allowed avidin, which presents a strong affinity for biotin, to be eliminated from the sample.

The concentrated sample was then injected on a TSK-gel filtration G3000SW exclusion chromatography (Tosoh Bioscience, Hampton, UK) pre-equilibrated in PBS buffer.

The major peak corresponding to OVAX was collected and concentrated on a concentration cell with 3500 Da exclusion (Millipore, UFC900324).

The different purification steps were analyzed by 4-20% SDS-PAGE gel in non-reducing conditions after staining with Coomassie Blue to check the purity of the protein.

1/2.2—Identifying OVAX by Mass Spectrometry.

The proteins were separated by SDS-PAGE in reducing conditions and stained with Coomassie Blue.

The major band of 45 kDa was extracted from the gel and rinsed with water and acetonitrile.

The proteins were reduced with dithiothreithiol, alkylated with iodoacetamide and digested by trypsin (12.5 ng/μL) (Roche Diagnostics GmbH, Mannheim, Germany) one night at 37° C. in 25 mM NH₄HCO₃ buffer, according to the protocol described by Shevchenko et al.

The peptide extracts were then dried.

Several mass spectrometry techniques were used to identify the OVAX and obtain maximum sequence coverage:
MALDI-TOF (Matrix Assisted Laser Desorption Ionisation-Time of Flight) mapping and
one-dimensional capillary liquid nano-chromatography coupled with tandem mass spectrometry (NanoLC-MS/MS).

Identification by the proteomics approach known as “bottom up” was performed on a peptide extract in solution in 8 μL of a 0.1% formic acid/2% acetonitrile mixture.

NanoLC-MS/MS analysis was performed using a CapLC system coupled to a “Quadrupole time-of-flight” (Q-TOF Ultima Global, Waters, Manchester, Great Britain) type of hybrid mass spectrometer equipped with a Z-spray ion source. The apparatus was calibrated with the help of a GluF solution at 500 fmol/μL (50% of a solution of 1% formic acid/50% acetonitrile, v/v). The samples were desalted and concentrated on-line by a precolumn (Monolithic trap column, 200 μm i.d×5 mm (PS-DVB), Dionex, ref 163972).

Peptide separation was carried out on a reversed-phase capillary column (Acclaim PepMap 100 C18, 5 μm, 75 μm I.D., 25 cm long, Dionex) with a flow-rate of 200 nL/min.

The gradient profile was as follows: Equilibration of the columns with 95% of solvent A (0.1% formic acid/2% acetonitrile/98% H2O, v/v) and 5% of solvent B (0.1% formic acid/20% H2O/80% acetonitrile, v/v), Gradient of 5 to 55% of B in 80 mins.; hold at 95% of B during 10 mins.

Data was acquired automatically between MS and MS/MS (fragmentation) modes: an MS survey scan was followed by three MS/MS scans on the three most intense peaks detected.

Only the di- and tri-charged ions were selected as precursors in an m/z mass range between 400 and 1,300.

The collision energy was selected according to the ion's mass and charge.

The raw data were processed by the ProteinLynx Global Server V 2.2.5 (PLGS) software system to create .pkl files comprising all the precursors selected in fragmentation as well as the list of associated fragment ions.

The sample was sonicated several minutes before depositing on the target.

1 μL of sample was deposited on the target with 1 μL of matrix according to the deposit method known as dried-drop.

The matrix used was a 5 mg/mL solution of CHCA (α-cyano-4-hydroxy-cinnamic acid) in 50% ethanol/50% acetonitrile/0.1% TFA/10 mM of 18-C-6 crown ether. The MALDI-TOF apparatus was calibrated externally with the help of peptides derived from the tryptic digestion of BSA (Bovine Serum Albumin) at 300 fmol/μL.

The MALDI-TOF spectra were obtained using a MALDI UR P/N mass spectrometer (Waters, Manchester, UK).

The analyses were performed in positive mode, in reflectron mode, with an acceleration voltage of 15 KV, pulse Voltage: 2,226, with a mass range (m/z) of 500 Da to 5,000 Da.

Computer processing of the spectra (substract, smooth, centroid) was performed with the help of the MassLynx V4.0 software system (Waters).

All the masses were revealed manually for all the mappings.

The experimental data from the MALDI and NanoLC-MS/MS analysis were compared to a non-redundant data bank (NCBInr 20071102, then 20110403) and with the help of the MASCOT software system (Matrix Science UK, http://www.matrixscience.com).

The enzyme specificity was established as trypsin, including two missing cleavages.

The following variable modifications were selected: carbamidomethyl cysteine and methionine oxidation.

The search was carried out in all taxonomies. The mass accuracy was set at ±0.3 Da. The candidate proteins were validated when the individual scores of the ions had a significance level greater than p<0.05.

1/2.3—Edman Sequencing

18 μg of OVAX were digested by trypsin (1.2 μM, Sigma-Aldrich, Saint-Quentin Fallavier) or chymotrypsin (1.2 μM, Sigma-Aldrich, Saint-Quentin Fallavier) in the presence of heparin (10 μg/mL, Sigma-Aldrich, Saint-Quentin Fallavier, France) during 1 hour at 37° C.

The proteins were separated by SDS-PAGE in reducing conditions and transferred onto polyvinylidene membrane.

The major band (40 kDa) was excised from the gel and analyzed by Edman sequencing.

The amino-terminal sequence of the protein was determined by automatic Edman degradation using the LF 3000 sequencer (Beckman/Porton) equipped with an HPLC system (“on-line Gold HPLC system”, Beckman Coulter) for detecting Phenylthiohydantoin-amino acid derivatives.

1/2.4—Predicting the OVAX Protein Sequence

The prediction of the OVAX sequence was performed from the gene's sequence available on the “Ensembl” site (http://www.ensembl.org/Gallus_gallus/Info/Index, ENSGALG00000019551), then subsequently from the sequence referenced in the NCBI database under number NW_{—}001471638.1, using the Genemark software system (Georgia Institute of Technology (http://exon.biology.gatech.edu/).

1/2.5—Analyzing the Antimicrobial Activity of OVAX

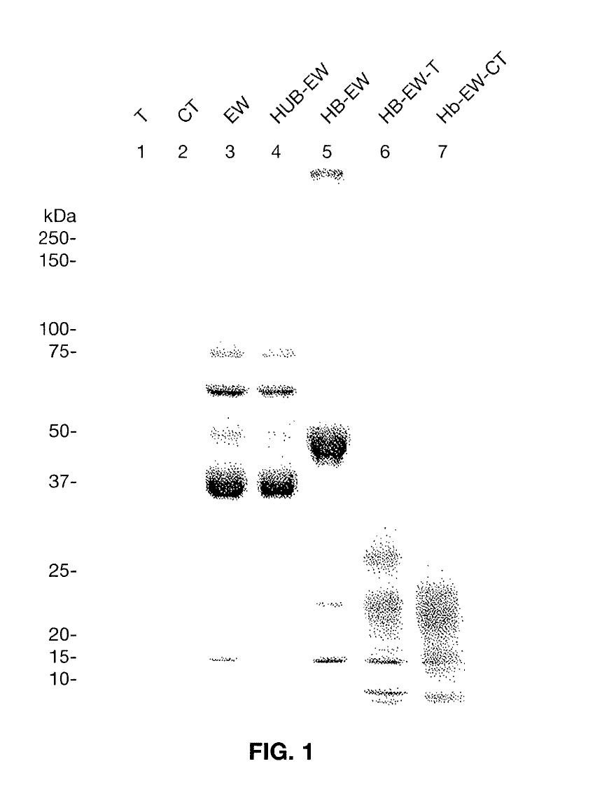

The samples of egg white (EW), of fractions not binding heparin (HUB-EW) and fractions binding heparin (HB-EW), were diluted in 50 mM Tris/150 mM NaCl buffer at pH 7.4, in order to obtain samples containing 1.8 mg/mL of proteins. The concentration of proteins was determined using the DC Protein test (Biorad, Saint-Quentin, France) with bovine serum albumin (Interchim, Montluçon, France) as control. The HB-EW samples (0.9 mg/mL) were digested either by trypsin at 5.5 μM (HB-EW-T) or by chymotrypsin at 1.2 μM (HB-EW-CT) in 50 mM Tris-HCL/150 mM NaCl, pH 7.4, buffer during 1 hour at 37° C. The reaction was stopped by cooling and the digestion products (5 μL for the trypsin, chymotrypsin, EW, HUB-EW, HB-EW samples, or 10 μL for the HB-EW-T and HB-EW-CT samples) were analyzed by 4-20% SDS-PAGE (FIG. 1). The antimicrobial activity of these various samples in a liquid medium was then analyzed.

The Listeria monocytogenes EGD bacterial strain was preserved by freezing in liquid nitrogen in the Brain-Heart Broth culture medium (Oxoid, Hampshire, England) containing 15% (v/v) glycerol.

The bacterial strain was cultivated for one night at 37° C. in Tryptone Soya broth (TSB, Oxoid), without agitation.

This preculture was then diluted and used to inoculate the culture media (TSB) so as to obtain an initial bacterial density of approximately 1.10⁷ CFU.mL⁻¹.

The culture was then incubated at 37° C. under agitation and the optical density was regularly measured at 600 nm until bacteria in half-exponential phase were obtained (approximately 1.10 ⁸ CFU.mL⁻¹ after approximately 5 hours of incubation).

In a first experiment, bacterial growth in the presence of different concentrations of Ovalbumin or OVAX (0-400 μg/mL) was monitored with the help of the Bioscreen C plate reader coupled with the Biolink software system (Oy Growth Curves Ab Ltd.) in the TSB culture medium.

The bacterial growth, sterility of the culture media and of the OVAX or Ovalbumin sample were checked on each plate.

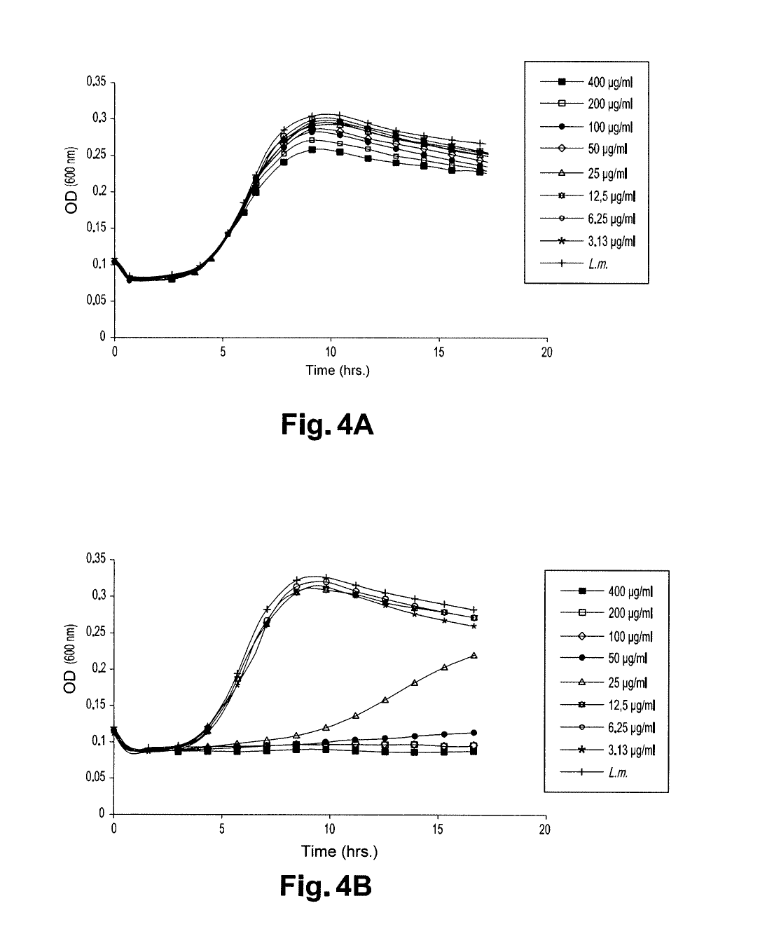

The bacterial growth was recorded over 24 hours continuously at 37° C., a reading of the optical density at 600 nm being performed every 45 minutes (FIG. 4).

In a second experiment, bacterial growth in the presence of different fractions of egg white was monitored with the help of the Bioscreen C plate reader coupled with the Biolink software system (Oy Growth Curves Ab Ltd.) in the TSB culture medium.

The bacterial growth, sterility of the culture media, of the fractions of egg white, trypsin and chymotrypsin were checked on each plate.

Each fraction of egg white (EW, HUB-EW, HB-EW, HB-EW digested by trypsin or chymotrypsin) was added in different concentrations. For the tests involving the fraction binding heparin after digestion by trypsin, increasing concentrations of trypsin were added, after two successive dilutions of the fraction of egg white binding heparin (HB-EW) to obtain a concentration of 0.9 μg/mL of HB-EW and 5.5 μM of trypsin.

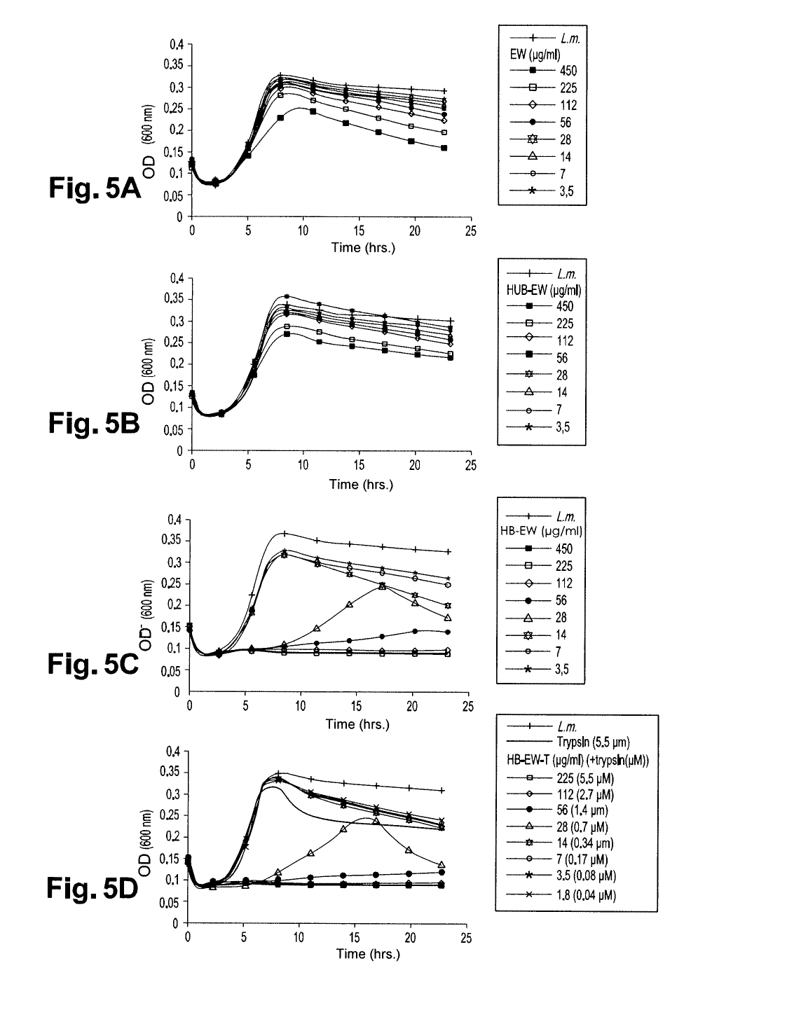

The bacterial growth was recorded over 24 hours continuously at 37° C., a reading of the optical density at 600 nm being performed every 45 minutes (FIG. 5).

1/3—Results

1/3.1—Purifying OVAX from Egg White

The two successive heparin-sepharose and biotin-agarose affinity chromatographies, followed by an exclusion chromatography, made it possible to eliminate the major proteins from the egg white and isolate the OVAX.

FIG. 1 is a photograph of a gel of the egg white proteins, at different levels of purification.

Thus, the third lane (EW) represents the migration of the egg white proteins as a whole.

The fifth lane (HB-EW) represents a fraction according to the invention, namely the fraction of egg white proteins and peptides binding heparin-sepharose. This first purification allows in particular the elimination of ovalbumin, a major protein in egg white (the fourth lane, HUB-EW, shows the egg white proteins not binding heparin). It appears that this fraction mainly contains a protein with a molecular weight of approximately 45 kDa.

The digestion of the fraction of interest by trypsin (Sixth lane—HB-EW-T) and by chymotrypsin (seventh lane—HB-EW-CT) shows that the protein of interest, with a molecular weight of approximately 45 kDa, corresponding to OVAX, is well cleaved into a plurality of peptides by these enzymes.

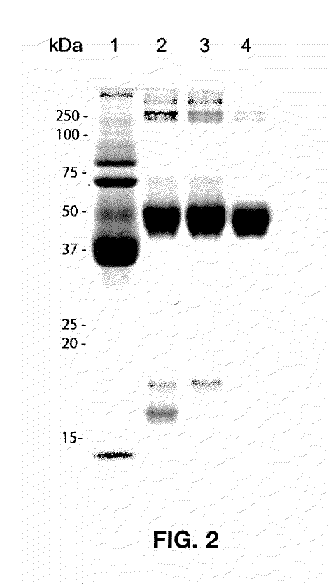

The same band at approximately 45 kDa was obtained during the successive steps of purifying the fraction of proteins and peptides derived from egg white and binding heparin, as can be seen in FIG. 2. Purification on heparin-sepharose (lane 2), followed by purification on biotin-agarose (lane 3) and filtration on gel (lane 4) allows the protein with molecular weight 45 kDa to be isolated.

Lane 4 represents a 92% pure protein, identified as being OVAX. The majority of contaminants of this fraction represent multimers of OVAX (high masses appearing on the gel). In effect, a western blot analysis showed that they react with anti-ovalbumin antibodies and disappear after reduction in the presence of beta-mercaptoethanol.

Thus, approximately 7.5 mg of pure OVAX proteins could be obtained from 50 mL of egg white.

The OVAX concentration is estimated to be a minimum of approximately 0.5 mg/mL of egg white, which makes it possible to envisage numerous applications, notably in the agri-food field, directly by purifying the protein from the egg white.

1/3.2—Identifying the OVAX

Identification by mass spectrometry and sequencing allowed the isolation and identification of a protein of sequence SEQ ID No. 1, corresponding to the OVAX isolated previously, which presents a sequence almost identical to the predicted sequence already known (SEQ ID No. 2) from the bioinformatic analysis of the chicken genome, and available in the NCBI data banks.

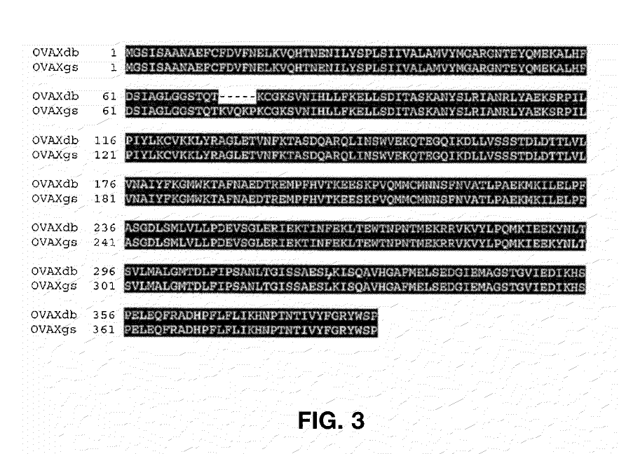

In effect, as can be seen in FIG. 3, which compares the two amino acid sequences, sequence SEQ ID No. 1 (OVAXgs) presents a difference on five amino acid residues compared to sequence SEQ ID No. 2 (OVAXdb).

These results were confirmed by amino terminal sequencing of the OVAX digested with trypsin.

More specifically, after proteolysis limited to trypsin, Edman sequencing (therefore by the N-terminal extremity) was performed, twice. The respective sequences VQKPKXGKSVNIHLLFXELL/VQKPXXGKSV were obtained, where X is a residue that could not be determined with certainty, which is often observed with cysteines.

Of the five residues (KVQKP) highlighted by Genemark, 4 (VQKP) could be confirmed. However, trypsin is known to specifically cut the peptide sequences after lysins (K) or arginines (R). This specificity tends to validate the residue K which precedes these four residues.

No Edman sequencing could be performed on the native OVAX, which suggests that it contains an amino-acetylation, blocking the N-terminal extremity of the molecule.

1/3.3—Antimicrobial Activity

The graphs in FIG. 4 allow the parallel to be drawn between the action over time of Ovalbumin (FIG. 4A) and OVAX (FIG. 4B) against Listeria monocytogenes, at increasing concentrations of these molecules, in liquid medium.

Thus, it can be seen that no specific action against Listeria monocytogenes was observed with Ovalbumin for concentrations below 400 μg/mL. The effect observed at 400 μg/mL is very weak.

In contrast it can be seen that OVAX presented an anti-Listeria monocytogenes activity from 25 μg/mL, at least during the first 10 hours of the test. With 50 μg/mL of OVAX, the anti-Listeria monocytogenes activity persisted beyond 15 hours.

The graphs in FIG. 5 allow the parallel to be drawn between the anti-Listeria monocytogenes activity of egg white (FIG. 5A—EW), the fraction of egg white not binding heparin (FIG. 5B—HUB-EW), the fraction of egg white binding heparin (FIG. 5C—HB-EW), the fraction of egg white binding heparin, after digestion by trypsin (FIG. 5D—HB-EW-T), and the fraction of egg white binding heparin, after digestion by chymotrypsin (FIG. 5E—HB-EW-CT).

The egg white, just like the fraction derived from egg white and not binding heparin, presented similar activity profiles. No significant anti-Listeria monocytogenes activity was observed, even at concentrations greater than 400 μg/mL.

In contrast, a similar anti-Listeria monocytogenes activity profile is observed for the fraction binding heparin and the fraction binding heparin digested by trypsin.

An intermediate action against Listeria monocytogenes was obtained from 28 μg/mL, this action being almost optimum at 56 μg/mL.

28 μg/mL of either of these fractions was sufficient to reduce growth of the bacterium by more than 50%. At this concentration, during the first 16 hours, the fractions HB-EW and HB-EW-T appear to interfere with the growth of bacteria and appear to act against this, bacterial growth being reduced by more than 50%. After 16 hours, a new degradation of the growth of the pathogen was observed.

The action of pure trypsin against Listeria monocytogenes was also studied (FIG. 5D). A slight effect was observed on the growth of Listeria monocytogenes. This effect has to be subtracted from that observed for OVAX at the highest concentration. In effect, in the most concentrated sample of OVAX, there was concentrated trypsin. This sample was then diluted in two-fold steps, the concentration of trypsin, as well as its own antimicrobial activity, thus being reduced in proportion. Here, trypsin acted as a control. Comparable results were obtained with the fraction binding heparin and digested by chymotrypsin (FIG. 5E).

These results are to be compared with those obtained with OVAX alone (FIG. 4B), which are similar. In these three cases, a dose-dependent effect of the fraction or molecule on Listeria monocytogenes was observed.

### EXPERIMENT 2

A second mode of purifying the OVAX protein was implemented in order to obtain the protein with a higher degree of purity. The antimicrobial activity thus obtained was tested in agar medium, as detailed below.

2/1—Collecting and Preparing the Egg White

The OVAX was purified from chicken egg white.

The freshly laid chicken eggs (ISA Brown, Hendrix Genetics, Saint-Brieuc, France) were collected, broken and the whites and yolks were sampled independently.

The egg whites were combined and the resulting mixture was diluted by ½ in 50 mM Tris-HCl/150 mM NaCl, pH 7.4 and homogenized. The mixture was then centrifuged at a 14,000 rpm at 4° C. during 10 minutes to eliminate the viscous proteins. The supernatant was kept at −20° C.

2/2—Purifying the OVAX

Purifying the OVAX from egg white was performed in three steps.

The first purification step was a Heparin-Sepharose type of affinity chromatography: 30 mL of the supernatant derived from egg whites prepared as described above were diluted by ½ in 50 mM Tris-HCl/300 mM NaCl, pH 7.4 and incubated overnight at 4° C. with beads of sepharose coupled with heparin (GE Healthcare, Uppsala, Sweden).

The beads of sepharose were washed extensively with a 50 mM Tris-HCl/150 mM NaCl, pH 7.4, buffer in order to eliminate the unbound peptides/proteins, and the affine proteins were then eluted with a 50 mM Tris-HCl/1M NaCl, pH 7.4, buffer.

The eluted fractions were concentrated on a concentration membrane (Millipore, Ireland, UFC800324) and the resulting sample was injected on a Sephacryl S-100 High Resolution, Hi-prep 16/60 column (GE Healthcare, Uppsala, Sweden) pre-equilibrated in 50 mM Tris-HCl/150 mM NaCl, pH 7.4, buffer.

The major peak containing OVAX was then passed through a biotin-sepharose type of affinity column, pre-equilibrated in 50 mM Tris-HCl/150 mM NaCl, pH 7.4, buffer, in order to eliminate any lane of avidin in the sample, the OVAX being recovered in the fraction not bound to the biotin beads.

The fraction not bound to the biotin beads, containing OVAX, was analyzed by SDS-PAGE electrophoresis in non-reducing conditions after staining with Coomassie blue.

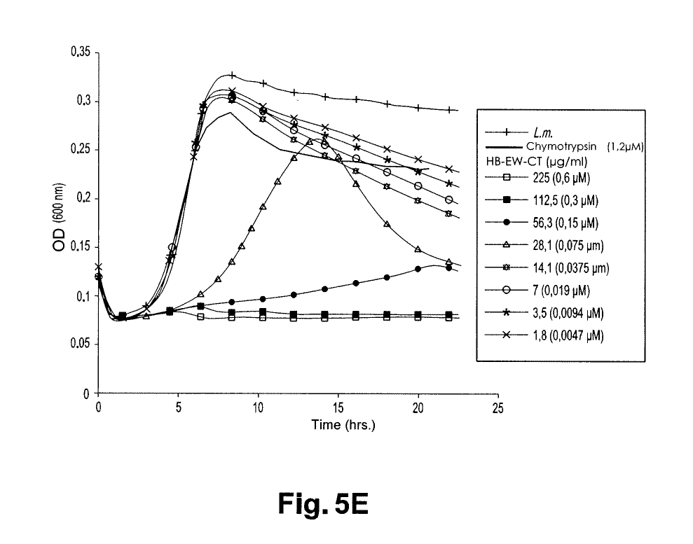

As shown in FIG. 6, the results show a heterogeneous band of apparent mass 40 to 50 kDa, the theoretical mass of OVAX being 44.2 kDa.

2/3—Identifying OVAX by Mass Spectrometry

5 μg (10 μL) of OVAX sample, purified as indicated above, were digested by trypsin during 16 hrs. at 37° C. in the following conditions.

10 μL of sample, 10 μL of 100 mM ammonium bicarbonate and 1 μL of 100 mM dithiothreithiol, were mixed and incubated during 30 mins. at 56° C.

1 μL of 250 mM iodoacetamide was added, and the solution was incubated during 30 mins. in the dark at ambient temperature.

2 μL of trypsin at 0.1 μg/μL in 0.01% TFA were added.

The peptides obtained by this digestion were analyzed by a CapLC nanoHPLC system (Waters, Manchester, UK) coupled to a Q-TOF Ultima Global mass spectrometer (Waters Micromass, Manchester, UK) equipped with a Z-spray ion source. The apparatus was calibrated with the help of a GluF solution at 500 fmol/μL (50% of a solution of 1% formic acid/50% acetonitrile, v/v).

The tubes containing the digestion products were brought to dry condition and 15 μL of buffer A (0.1% formic acid/2% acetonitrile/98% H₂O, v/v) were added. The samples were desalted and concentrated on-line by a precolumn (Monolithic trap column, 200 μm i.d×5 mm (PS-DVB), Dionex, ref 163972).

Peptide separation was carried out on a reversed-phase capillary column (Acclaim PepMap 100 C18, 5 μm, 75 μm I.D., 25 cm long, Dionex) with a flow-rate of 200 nL/min.

The gradient profile was as follows:
Equilibration of the columns with 95% of solvent A (0.1% formic acid/2% acetonitrile/98% H₂O, v/v) and 5% of solvent B (0.1% formic acid/20% H₂O/80% acetonitrile, v/v)
Gradient of 5 to 50% of B in 60 mins.
Hold at 95% of B during 10 mins.

Data was acquired automatically between MS and MS/MS (fragmentation) modes: an MS survey scan was followed by 4 MS/MS scans of the 4 most intense peaks detected.

The raw data were processed by the ProteinLynx Global Server V 2.2.5 (PLGS) software system to create .pkl files comprising all the precursors selected by fragmentation as well as the list of associated fragment ions.

The experimental data were compared to a data bank with the help of the MASCOT software system available on the local server.

The searches were carried out by selecting the following criteria:
data bank: nr NCBI
enzyme: trypsin
peptide charge: 2+ and 3+
2 miss cleavages
0.3 Da mass accuracy on MS and MS/MS
Data format: pkl
Instrument: ESI-QUAD-TOF
carbamidomethylation and oxidation of methionines in partial modifications
taxonomy: _Chordata_{—}

The results made it possible to identify OVAX (NCBI Reference Sequence: XP_{—}418984.2) with 6 unique peptides identified:

AGLETVNFK
ALHFDSIAGLGGSTQTK
QLINSWVEKQTEGQIK
SVNIHLLFKELLSDITASK
ILELPFASGDLSMLVLLPDEVSGLER
VQHTNENILYSPLSIIVALAMVYMGAR.

No other molecular species was identified in the sample.

These results indicate that the sample contained OVAX with a very high degree of purity (if not pure).

Edman sequencing, according to the method described above for Experiment 1, confirmed that this protein presents the amino-acid sequence SEQ ID No. 1.

2/4—Analyzing the Antimicrobial Activity of OVAX

The anti-Listeria monocytogenes activity of OVAX, purified as described above, was measured by the radial diffusion test according to the method described by Lehrer et al.

Beta-defensin 11 was used as a positive control (Hervé et al). The egg white ovalbumin was used as a negative control, the sequences of this protein and OVAX being very close (61% sequence identity) and their molecular weight being comparable (44.2 kDa for OVAX and 42.9 kDa for ovalbumin).

An H₂O control and a TBS NaCl 150 mM control, containing no peptide/protein, were also used.

The concentration of ovalbumin (Sigma-Aldrich, Saint-Quentin Fallavier, A7641) and OVAX was determined using the Biorad DC reagents (Biorad, Marnes la Coquette, France) and bovine serum albumin (Sigma-Aldrich, Saint-Quentin Fallavier, P0834) to establish the standard curve.

The bacteria (Listeria monocytogenes) were incubated all night under agitation at 37° C. The preculture obtained was then diluted in TSB (Tryptone Soya Broth) until the optical density measured at 600 or 620 nm was equal to 0.02 so as to standardize the culture conditions. The culture was incubated during 4 hrs. at 37° C. under agitation to obtain a culture in exponential growth phase. The bacteria were then centrifuged at 900 g during 10 mins. at 4° C., then washed once with a cold 10 mM sodium phosphate buffer (pH 7.4), and re-suspended in 10 ml of the same buffer. The optical density of this bacterial suspension was then measured to determine the volume to be collected that would make it possible to obtain 7.5.10⁶ cfu of bacteria. This last volume was mixed with 25 mL of “underlay” medium (10 mM sodium phosphate, pH 7.4, buffer containing 0.03% [weight/volume] BHI, 1% [weight/volume] agarose of low electroendosmosis [Sigma-Aldrich, St Quentin Fallavier, France] and 0.02% Tween-20) pre-heated to 46° C.

The agarose solution containing 7.5.10⁶ cfu of bacteria was poured into a petri dish to form a uniform 1 mm layer. 36 regularly spaced wells (3 mm in diameter) were formed in the agar gel with the help of a punch.

Solutions of OVAX, beta-defensin 11 and ovalbumin, in different concentrations, were produced.

5 μL of each of these solutions were deposited in two of the wells.

The petri dish was incubated at 37° C. during 3 hrs., to allow the molecules to diffuse in the agar medium containing the bacteria.

The first layer of agar medium (underlay) was then covered with 25 mL of “overlay” medium (10 mM sodium phosphate, pH 7.4, buffer containing 6% [weight/volume] BHI and 1% [weight/volume] agarose). After incubation for one night at 37° C., the diameter in mm of the inhibition zone around each well was determined as the difference between the diameter of the clear zone around the well and the diameter of the well. The larger this diameter, the more active the molecule against Listeria monocytogenes.

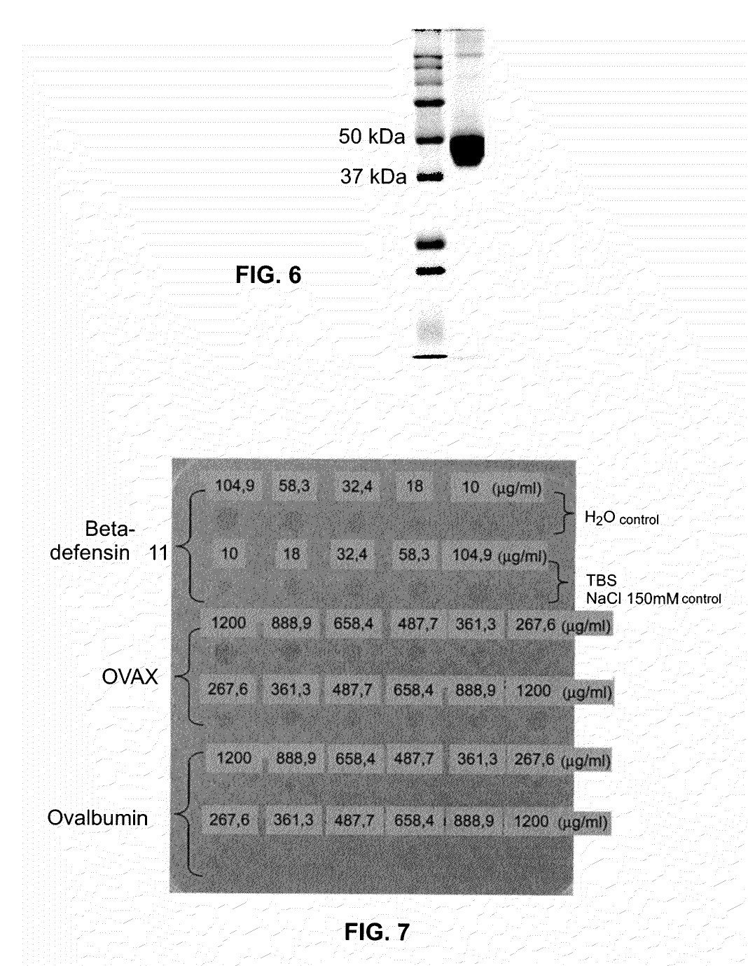

The results are shown in FIG. 7. Dose-dependent antimicrobial activity of the substantially pure OVAX with regard to Listeria monocytogenes can clearly be seen, whereas no activity is visible for ovalbumin.

### BIBLIOGRAPHIC REFERENCES

1. Benarafa, C., and E. Remold-O'Donnell. 2005. The ovalbumin serpins revisited: perspective from the chicken genome of clade B serpin evolution in vertebrates. Proc Natl Acad Sci USA 102:11367-72.
2. Heilig, R., R. Muraskowsky, C. Kloepfer, and J. L. Mandel. 1982. The ovalbumin gene family: complete sequence and structure of the Y gene. Nucleic Acids Res 10:4363-82.
3. Heilig, R., F. Perrin, F. Gannon, J. L. Mandel, and P. Chambon. 1980. The ovalbumin gene family: structure of the X gene and evolution of duplicated split genes. Cell 20:625-37.
4. Huntington, J. A., and P. E. Stein. 2001. Structure and properties of ovalbumin. J Chromatogr B Biomed Sci Appl 756:189-98.
5. Andersson, E., Rydengard, V., Sonesson, A., Morgelin, M., Bjorck, L. and Schmidtchen, A. (2004). Antimicrobial activities of heparin-binding peptides. Eur J Biochem 271, 1219-26.
6. Mine, Y. and Kovacks-Nolan, J. (2006). New insights in biologically active proteins and peptides derived from hen egg. World's Poultry Sci J 62, 87-95.
7. Proctor, V. A. and Cunningham, F. E. (1988). The chemistry of lysozyme and its use as a food preservative and a pharmaceutical. Crit Rev Food Sci Nutr 26, 359-95.
8. Mann, K. (2007). The chicken egg white proteome. Proteomics 7, 3558-68.
9. Shevchenko, A., Wilm, M., Vorm, O. and Mann, M. (1996). Mass spectrometric sequencing of proteins silver-stained polyacrylamide gels. Anal Chem 68, 850-8.
10. Cardin, A. D. and Weintraub, H. J. (1989). Molecular modeling of protein-glycosaminoglycan interactions. Arteriosclerosis 9, 21-32.
11. Margalit, H., Fischer, N. and Ben-Sasson, S. A. (1993). Comparative analysis of structurally defined heparin binding sequences reveals a distinct spatial distribution of basic residues. J Biol Chem 268, 19228-31.
12. Hughey, V. L. and Johnson, E. A. (1987). Antimicrobial activity of lysozyme against bacteria involved in food spoilage and food-borne disease. Appl Environ Microbiol 53, 2165-70.
13. Mann, K. (2008). Proteomic analysis of the chicken egg vitelline membrane. Proteomics 8, 2322-2332.
14 Lehrer et al. (1991). Ultrasensitive assays for endogenous antimicrobial polypeptides. J. Immunol. Methods. 137: 167-173
15. Hervé-Grépinet et al. (2010). Purification and characterization of avian beta-defensin 11 an antimicrobial peptide of the hen egg. Antimicrob. Agents Chemother. 54(10): 4401-4408

1. An isolated antimicrobial molecule having the amino-acid sequence SEQ ID No. 1.

2. A composition comprising the antimicrobial molecule according to claim 1 as active anti-Listeria monocytogenes ingredient, for its use as an anti-Listeria monocytogenes drug.

3. The composition according to claim 2, characterized in that the concentration of the antimicrobial molecule of sequence SEQ ID No. 1 is between 15 μg/mL and 400 μg/mL.

4. A food additive comprising the antimicrobial molecule according to claim 1 as an active anti-Listeria monocytogenes ingredient.

5. The food additive according to claim 4, characterized in that the concentration of the antimicrobial molecule of sequence SEQ ID No. 1 in the composition is between 15 μg/mL and 400 μg/mL.

6. An anti-Listeria monocytogenes composition comprising a fraction of peptides and/or proteins that are derived from egg white and that are capable of binding heparin, and that comprise at least the antimicrobial molecule of sequence SEQ ID No. 1.

7. The composition according to claim 6, characterized in that said fraction comprises in addition at least one molecule from amongst a protein similar to the protein MGC82112, Avidin, Lysozyme C, beta-defensin 11, the protein TENP, cyclophilin B, the protein Vmo-I, or Clusterin.

8. The composition according to claim 6, characterized in that said fraction comprises in addition at least one molecule from amongst Ovotransferrin, ovocleidin 17, ovoglycoprotein, ovalbumin, “plasma protease C1 inhibitor-like” or the “hypothetical protein” similar to pleitrophin.

9. The composition according to claim 6, characterized in that the antimicrobial molecule of sequence SEQ ID No. 1 represents a proportion of at least 50% of the fraction.

10. The composition according to claim 6, characterized in that the total concentration of peptides and/or proteins derived from egg white and capable of binding heparin is between 15 μg/mL and 400 μg/mL.

11. The composition according to claim 6, characterized in that the fraction is digested by at least one digestive enzyme.

12. The composition according to claim 11, characterized in that at least one digestive enzyme is a trypsin.

13. The composition according to claim 11, characterized in that at least one digestive enzyme is a chymotrypsin.

14. A method for inhibiting the growth of Listeria monocytogenes which comprises contacting Listeria monocytogenes with composition comprising a fraction of peptides and/or proteins that are derived from egg white and that are capable of binding heparin, and that comprise at least the antimicrobial molecule of sequence SEQ ID No. 1.

15. The method according to claim 14, characterized in that said fraction comprises in addition at least one molecule from amongst a protein similar to the protein MGC82112, Avidin, Lysozyme C, beta-defensin 11, the protein TENP, cyclophilin B, the protein Vmo-I, or Clusterin.

16. The method according to claim 14, characterized in that said fraction comprises in addition at least one molecule from amongst Ovotransferrin, ovocleidin 17, ovoglycoprotein, ovalbumin, “plasma protease C1 inhibitor-like” or the “hypothetical protein” similar to pleitrophin.

17. The method according to claim 14, characterized in that the antimicrobial molecule of sequence SEQ ID No. 1 represents a proportion of at least 50% of said fraction.

18. The method according to claim 14, characterized in that the total concentration of peptides and/or proteins derived from egg white and capable of binding heparin in the composition is between 15 μg/mL and 400 μg/mL.

19. The method according to claim 14, characterized in that the fraction is digested by at least one digestive enzyme.

20. The method according to claim 19, characterized in that the at least one digestive enzyme is a trypsin or a chymotrypsin.

21. (canceled)


SEQUENCE LISTING (2 sequences)

SEQ ID NO: 1 - protein, 393 aa, Gallus gallus
MGSISAANAEFCFDVFNELKVQHTNENILYSPLSIIVALAMVYMGARGNTEYQMEKALHFDSIAGLGGSTQTKVQKPKCGKSVNIHLLFKELLSDITASKANYSLRIANRLYAEKSRPILPIYLKCVKKLYRAGLETVNFKTASDQARQLINSWVEKQTEGQIKDLLVSSSTDLDTTLVLVNAIYFKGMWKTAFNAEDTREMPFHVTKEESKPVQMMCMNNSFNVATLPAEKMKILELPFASGDLSMLVLLPDEVSGLERIEKTINFEKLTEWTNPNTMEKRRVKVYLPQMKIEEKYNLTSVLMALGMTDLFIPSANLTGISSAESLKISQAVHGAFMELSEDGIEMAGSTGVIEDIKHSPELEQFRADHPFLFLIKHNPTNTIVYFGRYWSP

SEQ ID NO: 2 - protein, 388 aa, Gallus gallus
MGSISAANAEFCFDVFNELKVQHTNENILYSPLSIIVALAMVYMGARGNTEYQMEKALHFDSIAGLGGSTQTKCGKSVNIHLLFKELLSDITASKANYSLRIANRLYAEKSRPILPIYLKCVKKLYRAGLETVNFKTASDQARQLINSWVEKQTEGQIKDLLVSSSTDLDTTLVLVNAIYFKGMWKTAFNAEDTREMPFHVTKEESKPVQMMCMNNSFNVATLPAEKMKILELPFASGDLSMLVLLPDEVSGLERIEKTINFEKLTEWTNPNTMEKRRVKVYLPQMKIEEKYNLTSVLMALGMTDLFIPSANLTGISSAESLKISQAVHGAFMELSEDGIEMAGSTGVIEDIKHSPELEQFRADHPFLFLIKHNPTNTIVYFGRYWSP